(19) United States
(12) Patent Application Publication
(10) Pub. No.: US 2010/0158990 A1
(43) Pub. Date: Jun. 24, 2010

(54) TRANSDERMAL METHOD AND PATCH FOR CORTICOSTEROID ADMINISTRATION

(75) Inventors: Suresh Borsadia, Plainsboro, NJ (US); Kalpana Patel, West Windsor, NJ (US)

Correspondence Address: RAYMOND R. MOSER JR., ESQ.;MOSER IP LAW GROUP, 1030 BROAD STREET, 2ND FLOOR, SHREWSBURY, NJ, 07702, US

(73) Assignee: ABIELLE PHARMACEUTICALS, INC, Princeton, NJ (US)

(21) Appl. No.: 12/521,064

(22) PCT Filed: Dec. 26, 2007
(86) PCT No.: PCT/US07/88807

§ 371 Date: Jan. 27, 2010

Related U.S. Application Data
(60) Provisional application No. 60/882,039, filed on Dec. 27, 2006.

(51) Int. Cl. A61K 9/70; A61K 31/573; A61P 29/00; A61P 9/10; A61P 1/08; A61P 35/00; A61P 7/10; A61P 31/00; A61P 11/06

(52) U.S. Cl. 424/448; 514/180

(57) ABSTRACT

A method of treating an individual having a disease or for diagnostic purposes requiring administration of a corticosteroid, which involves applying to a portion of intact derma on the individual a transdermal patch having a backing layer and a matrix adhesive layer. The matrix adhesive layer includes a skin-compatible pressure-sensitive adhesive, a corticosteroid, and at least one permeation enhancer. The matrix adhesive layer, when applied to the skin of the individual, transdermally and continuously delivers the corticosteroid to the mammal for systemic treatment of the disease or for the diagnostic purpose.

The present invention relates to a transdermal method and device for the administration of a corticosteroid for the systemic treatment of a disease or for diagnostic purposes in an individual. More particularly, the invention relates to a transdermal method and patch for the administration of dexamethasone for the systemic treatment of a disease or for a diagnostic purpose.

Dexamethasone is an example of a corticosteroid that may be administered to treat inflammation; myocardial infarction; nausea and vomiting, including nausea and vomiting due to chemotherapy, radiotherapy, or other pharmaceutical treatment; cancer treatment; perioperative and post-operative trauma, injury, and edema; ecchynosis; respiratory tract infections; asthma; other diseases and for other diagnostic purposes. Common methods of corticosteroid administration include oral, such as tablets and liquids, and parenteral, such as by intramuscular or intravenous administration or injection into a joint space. In cases of localized inflammation, such as with arthritis, or of itching and skin irritation, such as with poison ivy rash, topical corticosteroids can by applied locally to the affected area to provide local treatment of the condition.

Many of the diseases and diagnostic purposes that are most effectively treated with corticosteroids, however, require ongoing treatment over the course of two or more days. Additionally, the nature of many of these diseases and diagnostic purposes makes effective oral administration of medication uncomfortable and difficult, if not impossible. For instance, in any situation where a patient is suffering from nausea and vomiting, oral administration of a drug is challenging and creates more discomfort for the patient. Some patients may also find that oral administration of these drugs causes gastric discomfort or irritation. Administration by injection is generally impractical for home use.

The delivery of drugs through the derma, i.e. skin and mucosa, provides many advantages over other routes of administration. Primarily, transdermal drug delivery is a comfortable, convenient, and noninvasive way of administering drugs. The variable rates of absorption and metabolism associated with oral administration are avoided, as are other inherent inconveniences such as gastrointestinal irritation and the like. Transdermal drug delivery also makes possible a high degree of control over blood concentrations of any particular drug and allows for consistent drug delivery. These advantages enhance patient compliance and improve the safety and efficacy of medications.

Skin is a structurally complex, relatively thick membrane. Molecules moving from the environment into and through the intact skin must first penetrate the stratum corneum and any material on its surface. Such molecules must then penetrate the viable epidermis, the papillary dermis, and the capillary walls into the bloodstream or lymph channels for systemic treatment. To be so absorbed, molecules must overcome a different resistance to penetration in each type of tissue. Additionally, each type of drug molecule may present its own unique characteristics relating to penetration in each type of tissue. Transport across the skin membrane is thus a complex phenomenon such that no single formulation can be applied for different drugs. While a number of writings on transdermal delivery discuss delivery of steroids, the majority of these are enabled for the delivery of steroids effective when delivered in minute quantities per day, such as estrogen, progestin and the like. These formulations will thus not be amenable to the delivery of corticosteroids which are administered in milligram doses. Additionally, some of the known transdermal delivery formulations containing steroid drugs indicate pH-sensitivity and include only components that are free of acid groups.

Devices for transdermal administration of drugs generally fall into either the category of liquid reservoir patches or of matrix patches. In liquid reservoir patches, the drug is stored as a liquid in a reservoir from which it diffuses to the skin. The patch includes a boundary layer that may include a rate-controlling membrane to control the release rate of the drug. In matrix patches, the drug is stored in a polymeric matrix that can be made of one or more layers for storing the drug, controlling the rate of release, and adhering to the skin. Liquid reservoir patches are easier to develop than matrix patches because of fewer problems such as incompatibility of drug and polymeric materials. Matrix patches, however, are easier to manufacture than liquid reservoir patches and are more comfortable and convenient to wear.

Thus, it is desirable to provide a transdermal method and device, such as a matrix patch, for the administration of a corticosteroid for the systemic treatment of a disease or for diagnostic purposes.

### SUMMARY OF THE INVENTION

The present invention relates to a transdermal method and device for the administration of a corticosteroid for the systemic treatment of a disease or for diagnostic purposes in an individual. More particularly, the invention relates to a transdermal matrix patch and method of using it for the administration of dexamethasone for the systemic treatment of a disease or for diagnostic purposes in an individual.

In one embodiment, the invention relates to a method of treating an individual with a disease or for diagnostic purposes requiring the systemic administration of a corticosteroid by applying to intact skin of the individual a transdermal matrix patch having a backing layer and a matrix adhesive layer with a skin-compatible pressure-sensitive adhesive, a corticosteroid, and at least one permeation enhancer. The matrix adhesive layer, when applied to the intact skin of the individual, transdermally and continuously delivers the corticosteroid to the individual for systemic treatment of a disease or for diagnostic purposes. In one embodiment, the corticosteroid is dexamethasone. In one embodiment, the permeation enhancer is selected from fatty acids, esters of fatty acids, straight, cyclic or branched chain alkyl alcohols and their esters and ethers and combinations of these.

In a further embodiment, the invention relates to a transdermal matrix patch having a backing layer and a matrix adhesive layer with a skin-compatible acrylic adhesive, a corticosteroid in an amount sufficient to deliver a therapeutically effective daily dose, and at least one permeation enhancer selected from fatty acids, esters of fatty acids, straight or branched chain alkyl alcohols and their esters, and combinations of these. In one embodiment, the permeation enhancer is present in the matrix adhesive layer in an amount between 0.5% and 15% based on the total weight of the matrix adhesive layer. When the matrix patch is applied to the skin of an individual, the matrix adhesive layer transdermally and continuously delivers—over an extended period—the corticosteroid to the individual for systemic treatment of a disease or for diagnostic purposes. In one embodiment, the matrix patch delivers a daily dose of between 0.25 and 8.0 mg of the corticosteroid such as dexamethasone. In one embodiment, the permeation enhancer is selected from oleic acid, isopropyl myristate, lauryl alcohol, dimethyl isosorbide, methyl oleate, and combinations of these. In one embodiment, the permeation enhancer is selected from fatty acids, fatty acid esters, C8-C16 alcohols (such as straight-chain alcohols), polyhydric alcohols whose hydroxyls are or are not alkylated with C1-C4 alkyls, cyclic or bicyclic compounds of C/H/O having 5 to 10 ring atoms of carbon or oxygen and two or more hydroxyls whose hydroxyls are or are not alkylated with C1-C4 alkyls, branched compounds of C/H/O having 5 to 10 backbone atoms of carbon or oxygen and two or more hydroxyls whose hydroxyls are or are not alkylated with C1-C4 alkyls, or mixtures thereof. The fatty acid or fatty acyl moieties can be, for example C12-C30, or C14, and higher, or C16 and higher, or C24 and lower, or C22 or lower, or C20 or lower, or C18 or lower. The fatty acid or fatty acyl moieties can include one or more unsaturations, which can for example provide cis-double bonds. The alcohol moieties of fatty acid esters can be, for example, C1-C6, or C2-C4, such as for example isopropanol.

Surprisingly the present invention shows higher flux from compositions containing lower amounts of the active ingredient. This rate of delivery is further enhanced by an optimum level and/or combination of permeation enhancer.

### DETAILED DESCRIPTION

As used herein, the term “corticosteroid” refers to any of the steroid hormones produced by the adrenal cortex or their synthetic equivalents.

As used herein, the terms “disease” and “disease state” refer to any condition of an individual characterized by specific signs and symptoms associated with sickness or illness or vital function impairment.

As used herein, the term “diagnostic” refers to a product or process used for the purpose of detecting and identifying a disease state, condition, or normal function in an individual.

As used herein, the term “individual” refers to a living mammal and includes, without limitation, humans and other primates, livestock and sports animals such as cattle, pigs and horses, and pets such as cats and dogs.

As used herein, the term “matrix adhesive layer” refers to a drug intimately admixed, i.e. dissolved or suspended, in a biocompatible adhesive phase, preferably a pressure sensitive adhesive, which can also contain other ingredients.

As used herein, the term “permeation enhancement” refers to an increase in the permeability of skin to a therapeutic agent in the presence of a permeation enhancer as compared to permeability of skin to the same therapeutic agent in the absence of a permeation enhancer.

As used herein, the term “permeation enhancer” refers to an agent or a mixture of agents which acts to increase the permeability of the skin to therapeutic agents.

As used herein, the term “permeation-enhancing amount” refers to an amount of a permeation enhancer which provides permeation enhancement throughout a substantial portion of the administration period.

As used herein, the phrase “portion of intact derma” refers to a defined area of intact unbroken skin or mucosal tissue. That area will usually be in the range of about 5 cm² to about 100 cm².

As used herein, the term “transdermal” refers to both percutaneous and transmucosal administration, i.e., passage of a drug, such as a corticosteroid through a body surface or membrane such as intact unbroken skin or intact unbroken mucosal tissue into the systemic circulation.

As used herein, the term “percutaneously absorbable” refers to the ability of a drug to pass through a body surface or membrane such as intact unbroken skin or mucosal tissue into the systemic circulation when formulated in a transdermal device of the invention.

As used herein, the term “skin-contacting layer” is a layer of a transdermal device for contacting skin or mucosa.

As used herein, the term “therapeutically effective dose” refers to an amount of a drug required on average by an appropriate collection of individuals to provide a desired therapeutic effect. It will be understood that a diagnostic dose shall be modeled on a therapeutic target. Thus, a therapeutically effective dose is a diagnostically effective dose.

The present invention relates to a method of treating a disease or for a diagnostic purpose requiring the administration of a corticosteroid by transdermally administering the corticosteroid. Examples of corticosteroids for use in the present invention include, without limitation, hydroxycortisone, cortisone, desoxycorticosterone, fludrocortisone, betamethasone, dexamethasone, prednisolone, prednisone, methylprednisolone, paramethasone, triamcinolone, flumethasone, fluocinolone, fluocinonide, fluprednisolone, halcinonide, flurandrenolide, meprednisone, medrysone, and esters and mixtures thereof. The present invention also relates to devices and compositions for use with the method of the invention.

In certain embodiments, the method of the present invention is useful for treating disease or for diagnostic purposes requiring the administration of dexamethasone, including without limitation, myocardial infarction, inflammation, asthma, ecchynosis, perioperative and postoperative trauma and injury, edema, certain cancers, respiratory tract infections, and nausea and vomiting.

In certain embodiments the method of the present invention may find particular usefulness in treating nausea and vomiting due to chemotherapy, radiation therapy, other drug therapy, motion sickness, or post-operative reaction. Because this method involves the transdermal and continuous administration of a corticosteroid, even over the course of several days, it is effective in both preventing, ameliorating or treating nausea and vomiting for an extended time and without exacerbating existing nausea and vomiting by oral ingestion. Additional benefits of the present invention include improved patient compliance, since the method involves the placement of a transdermal device, which in some embodiments is left in place for 3 days or more; patient protection from nausea and vomiting from the time the device is applied until it is removed; increased patient confidence to leave the hospital or doctor's office after chemotherapy, knowing that the device will prevent or reduce nausea and vomiting. Additionally, the device delivers a therapeutically effective daily dose of the corticosteroid for the entire treatment period or until it is removed. Since the device delivers the corticosteroid at a continuous and controlled rate, there is no initial spike in plasma concentration as when the drug is administered by IV; therefore, the method reduces side effects that may sometimes be experienced with other forms of administration.

In certain embodiments, the method of the invention may find particular usefulness in treating asthma or other respiratory disorders. Individuals who are affected by these conditions may find breathing to be difficult because of inflamed and swollen airways. Systemic corticosteroid treatment may be prescribed for the treatment of these conditions. However, individuals who have difficulty breathing because of these conditions may often find swallowing an oral dosage form of any drug difficult and uncomfortable. IM and IV injections are impractical for home use; therefore, a method of transdermally administering a corticosteroid for systemic treatment of asthma and other respiratory disorders helps to reduce patient discomfort of drug administration while delivering a continuous and controlled dosage of the drug.

In certain embodiments, the method of the invention may find particular usefulness in diagnosing certain conditions, such as, for example, Tolosa-Hunt Syndrome or wherever dexamethasone suppression test is used.

In certain embodiments, the device of the invention provides a substantially consistent flux rate of dexamethasone of 0.1 μg/cm²/hour or more for 24 hours or more, 48 hours or more, or 72 hours or more. In certain embodiments, the device of the invention provides a consistent flux rate of dexamethasone of 0.2 μg/cm²/hour or more for 24 hours or more, 48 hours or more, or 72 hours or more.

In certain embodiments, the device of the invention transdermally provides a therapeutically effective daily dose of corticosteroid. In certain embodiments, the device of the invention transdermally provides a therapeutically effective daily dose, such as, for example, between 0.25 and 8 mg/day, of dexamethasone. In certain embodiments, the device provides such a dose for 2 days or more, or 3 days or more.

One embodiment of the device of the present invention is a transdermal patch for application to the skin or mucosa of an individual. The patch has a skin-contacting matrix adhesive layer laminated or otherwise attached to a backing layer. Typically, the matrix adhesive layer is covered by a removable release liner before use to protect the matrix adhesive surface and keep it clean until it is applied to the skin or mucosa.

The backing layer acts as a support for the matrix adhesive layer and provides a barrier layer that prevents loss of the drug in the matrix adhesive layer to the environment. The material chosen for the backing should be compatible with the adhesive, drug, and permeation enhancer, and should be minimally permeable to any components of the patch. The backing can be opaque to protect components of the matrix patch from degradation from exposure to ultraviolet light. Further, the backing should be capable of binding to and supporting the adhesive layer, yet should be pliable to accommodate the movements of a person using the patch. Suitable materials for the backing include metal foils, metalized polyfoils, composite foils or films containing polyester such as polyester terephthalate, polyester or aluminized polyester, polytetrafluoroethylene, polyether block amide copolymers, polyethylene methyl methacrylate block copolymers, polyurethanes, polyvinylidene chloride, nylon, silicone elastomers, rubber-based polyisobutylene, styrene, styrene-butadiene and styrene-isoprene copolymers, polyethylene, and polypropylene. A thickness of about 0.0005 to 0.01 inch can, for example, be used. As is known in the art, adhesive monomers can include carboxylic acid moieties (or salts thereof) and/or other functional groups, such as hydroxyl. Or, adhesive monomers may have no functional monomers (as synthesized, assuming no substantial hydrolysis of, for example, ester linkages). Adhesive polymers are often crosslinked to some degree, such as by use of crosslinking monomer.

Useful adhesives include, for example, acrylics (e.g., polyacrylates including alkyl acrylates), polyvinyl acetates, natural and synthetic rubbers, ethylenevinylacetate copolymers, polysiloxanes, polyurethanes, plasticized polyether block amide copolymers, plasticized styrene-butadiene rubber block copolymers, and mixtures thereof. Polyacrylates can be, for example, Duro-Tak 87-4098, Duro-Tak 87-2052, Duro-Tak 387-2353 (or Duro-Tak 87-2353), Duro-Tak 387-2287 (or Duro-Tak 87-2287), Duro-Tak 387-2516 (or Duro-Tak 87-2516) (all from National Starch & Chemical, Bridewater, N.J.), or mixtures thereof. Styrene-butadiene rubber pressure sensitive adhesive can be, for example, DURO-TAK® 87-6173 adhesive (National Starch & Chemical).

The release liner can be made of the same materials as the backing, or other suitable films coated with an appropriate release surface.

The patch can further comprise various additives in addition to the adhesive, corticosteroid, and permeation enhancer. These additives are generally those pharmaceutically acceptable ingredients that are known in the art of drug delivery and, more particularly, in the art of transdermal drug delivery. Nonlimiting examples of additive ingredients include diluents, excipients, emollients, plasticizers, skin irritation reducing agents (which can also include agents that reduce irritation to mucosa), carriers, and mixtures of these. For example, suitable diluents can include mineral oil, low molecular weight polymers, plasticizers, and the like. Some transdermal drug delivery formulations have a tendency to cause irritation after prolonged exposure to the skin or mucosa, thus addition of an irritation reducing agent aids in achieving a composition that is better tolerated by the skin or mucosa.

For transdermal delivery of a corticosteroid according to an embodiment of the method of the present invention, a transdermal patch with a matrix adhesive layer containing a skin-compatible pressure-sensitive adhesive, a corticosteroid, and a permeation enhancer is applied to the intact derma of an individual. The patch transdermally and continuously delivers corticosteroid to the individual for systemic treatment of a disease or for diagnostic purposes requiring the administration of a corticosteroid.

In certain embodiments, the amount of corticosteroid(s) in the adhesive layer, by weight of the adhesive layer, is 15% or less, or 14% or less, or 13% or less, or 12% or less, or 11% or less, or 10% or less, or 9% or less, or 8% or less, or 7% or less, or 6% or less, or 5% or less, or 4% or less. In certain embodiments, the amount of corticosteroid(s) in the adhesive layer, by weight of the adhesive layer, is 0.25% or more, 0.5% or more, 1% or more, or 2% or more, or 3% or more, or 4% or more, or 5 or more, or 6% or more, or 7% or more, or 8% or more.

In certain embodiments, the amount of permeation enhancer(s) in the adhesive layer, by weight of the adhesive layer, is 15% or less, or 14% or less, or 13% or less, or 12% or less, or 11% or less, or 10% or less, or 9% or less, or 8% or less, or 7% or less, or 6% or less, or 5% or less, or 4% or less, or 3% or less. In certain embodiments, the amount of permeation enhancer(s) in the adhesive layer, by weight of the adhesive layer, is 0.5% or more, 1% or more, or 2 or more, or 3% or more, or 4 or more, or 5 or more, or 6 or more.

### Example 1

### Preparation of Samples

TABLE 1

| Sample ID | Acrylic Adhesive | Dexamethasone |
|---|---|---|
| 5% Dex/87-900A | 95% DURO-TAK 87-900A | 5% |
| 10% Dex/87-900A | 90% DURO-TAK 87-900A | 10% |
| 15% Dex/87-900A | 85% DURO-TAK 87-900A | 15% |
| 5% Dex/87-2510 | 95% DURO-TAK 87-2510 | 5% |
| 10% Dex/87-2510 | 90% DURO-TAK 87-2510 | 10% |
| 15% Dex/87-2510 | 85% DURO-TAK 87-2510 | 15% |

The acrylic adhesives used in Example 1 were DURO-TAK® 87-900A (no functional monomers, no vinyl acetate) and DURO-TAK® 87-2510 (hydroxyl functional), both available from National Starch and Chemical in Bridgewater, N.J. The release liner used in the examples herein is polyester and is available from Loparex, Inc. The backing used in the examples herein is also polyester and is available from 3M as 2610F. Samples were prepared according to the amounts shown in Table 1. The dexamethasone was mixed into the acrylic adhesive to form a uniform suspension. The suspension was coated onto the siliconized surface of the polyester release liner to the desired thickness. The coated release liner was then placed in a drying oven to remove the solvents. The dry adhesive-coated release liner was then laminated with the polyester backing layer. The multi-layer laminate was cut by punching out units of the desired size and geometry for delivery of the desired target daily dose. Alternatively, the laminate may be wound into rolls for storage or transport to another location. The rolled laminate may then be unwound and cut by punching out units of the desired size and geometry. These punched units were then placed in individual pouches and sealed for later use as patches.

### Example 2

### In Vitro Flux of Dexamethasone from Transdermal Delivery Device

Heat-separated human cadaver skin was cut to the desired size and mounted on a Franz diffusion cell. The release liner was peeled away from a patch made as described in Example 1 above. The patch was placed on the skin and the patch and skin were clamped together. Receptor solution was added to the diffusion cell, and the assembly was maintained at 32° C. Aliquots of the receptor solution were taken at periodic time points (2 hours, 4 hours, 10 hours, 24 hours, 48 hours, 72 hours, 96 hours, and 120 hours). The concentration of the dexamethasone in the receptor solution was measured at each time point, and the cumulative delivery of dexamethasone over the indicated time was calculated. The results are shown in Tables 2 and 3.

TABLE 2
Cumulative in vitro transdermal flux of dexamethasone from DURO-TAK 87-900A matrix
(In μg/cm²)

| Sample ID | 0 hr. | 2 hr. | 4 hr. | 10 hr. | 24 hr. | 48 hr. | 72 hr. | 96 hr. | 120 hr. |
|---|---|---|---|---|---|---|---|---|---|
| 5% Dex/ | 0 | 0.9 ± 0.3 | 1.9 ± 0.2 | 3.8 ± 0.1 | 11.4 ± 2.1 | 20.0 ± 3.3 | 30.5 ± 5.0 | 38.9 ± 6.3 | 46.5 ± 7.6 |
| 87-900A | | | | | | | | | |
| 10% Dex/ | 0 | 0.4 ± 0.1 | 0.7 ± 0.3 | 1.5 ± 0.7 | 4.2 ± 1.8 | 7.6 ± 2.8 | 12.2 ± 3.6 | 17.8 ± 2.1 | 23.1 ± 1.0 |
| 87-900A | | | | | | | | | |
| 15% Dex/ | 0 | 0.2 ± 0.2 | 0.5 ± 0.2 | 1.0 ± 0.5 | 4.5 ± 4.1 | 5.5 ± 3.7 | 9.0 ± 5.8 | 13.3 ± 8.7 | 17.0 ± 11.4 |
| 87-900A | | | | | | | | | |

TABLE 3
Cumulative in vitro transdermal flux of dexamethasone from DURO-TAK 87-2510 matrix
(In μg/cm²)

| Sample ID | 0 hr. | 2 hr. | 4 hr. | 10 hr. | 24 hr. | 48 hr. | 72 hr. | 96 hr. | 120 hr. |
|---|---|---|---|---|---|---|---|---|---|
| 5% Dex/ | 0 | 0.6 ± 0.1 | 1.3 ± 0.4 | 2.3 ± 0.5 | 7.5 ± 3.1 | 13.6 ± 5.0 | 21.6 ± 7.3 | 29.2 ± 9.4 | 36.4 ± 10.5 |
| 87-2510 | | | | | | | | | |
| 10% Dex/ | 0 | 0.4 ± 0 | 0.6 ± 0.2 | 1.2 ± 0.7 | 3.6 ± 1.8 | 6.5 ± 3.5 | 10.9 ± 5.6 | 15.7 ± 8.0 | 19.6 ± 9.7 |
| 87-2510 | | | | | | | | | |
| 15% Dex/ | 0 | 0 ± 0 | 0.3 ± 0.1 | 0.6 ± 0.2 | 1.5 ± 0.5 | 2.6 ± 1.0 | 4.3 ± 1.6 | 6.2 ± 2.3 | 7.7 ± 2.6 |
| 87-2510 | | | | | | | | | |

The results show, surprisingly, that the lower concentrations of dexamethasone in the adhesive matrix provide higher in vitro flux. This is true for both of the acrylic adhesives tested.

### Example 3

### Preparation of Samples Including Permeation Enhancers

TABLE 4

| | Acrylic | Dexa- | Oleic | Lauryl | Dimethyl |
|---|---|---|---|---|---|
| Sample ID | Adhesive | methasone | Acid | Alcohol | Isosorbide |
| 5% Dex | 95% | 5% | — | — | — |
| 5% Dex/3% OA | 92% | 5% | 3% | — | — |
| 5% Dex/3% OA/ | 90% | 5% | 3% | 2% | — |
| 2% LA | | | | | |
| 5% Dex/3% OA/ | 90% | 5% | 3% | — | 2% |
| 2% DMI | | | | | |

Samples were prepared as described in Example 1 above using DURO-TAK 87-900A adhesive and other components in the amounts shown in Table 4. In this example, 5% dexamethasone was used for all samples, and selected permeation enhancers, i.e. oleic acid, lauryl alcohol, and dimethyl isosorbide, were also mixed into the acrylic adhesive to form a uniform suspension.

### Example 4

### In Vitro Transdermal Flux of Dexamethasone from Transdermal Delivery Device in the Presence of Permeation Enhancers

In vitro flux testing was conducted on the samples of Example 3 in the same manner as the in vitro flux testing described above in Example 2, with aliquots of the receptor solution being taken at 25 hours, 48 hours, 72 hours, and 96 hours. The results are shown in Table 5.

TABLE 5
Cumulative in vitro transdermal flux of dexamethasone from DURO-TAK 87-900A
(In μg/cm²)

| Sample ID | 0 hours | 25 hours | 48 hours | 72 hours | 96 hours |
|---|---|---|---|---|---|
| 5% Dex | 0 | 17.4 ± 4.7 | 31.2 ± 8.3 | 45.0 ± 13.0 | 57.0 ± 16.0 |
| 5% Dex/3% OA | 0 | 24.4 ± 3.9 | 46 ± 8.7 | 63.0 ± 10.0 | 78.0 ± 12.0 |
| 5% Dex/3% OA/ | 0 | 20.1 ± 4.7 | 37.1 ± 7.7 | 51.3 ± 9.0 | 65.0 ± 7.0 |
| 2% LA | | | | | |
| 5% Dex/3% OA/ | 0 | 20.8 ± 4.2 | 37.9 ± 3.9 | 53.0 ± 5.0 | 67.0 ± 5.0 |
| 2% DMI | | | | | |

Dex: Dexamethasone; OA is Oleic Acid; LA is Lauryl Alcohol and DMI is dimethyl isosorbide.

These results show that the patches made according to Example 3 transdermally and continuously deliver the dexamethasone from the matrix adhesive layer, and the addition of permeation enhancers increases the cumulative flux of the dexamethasone.

Publications and references, including but not limited to patents and patent applications, cited in this specification are herein incorporated by reference in their entirety in the entire portion cited as if each individual publication or reference were specifically and individually indicated to be incorporated by reference herein as being fully set forth. Any patent application to which this application claims priority is also incorporated by reference herein in the manner described above for publications and references.

While this invention has been described with an emphasis upon certain embodiments, it will be obvious to those of ordinary skill in the art that variations in the preferred devices and methods may be used and that it is intended that the invention may be practiced otherwise than as specifically described herein. Accordingly, this invention includes all modifications encompassed within the spirit and scope of the invention as defined by the claims that follow.

What is claimed:

1. A method of treating an individual having a disease or for diagnostic purposes requiring administration of a corticosteroid, the method comprising:
providing a transdermal patch comprising:
a. a backing layer; and
b. a matrix adhesive layer comprising:
i. a skin-compatible pressure-sensitive adhesive;
ii. a corticosteroid; and
iii. at least one permeation enhancer;
applying the transdermal patch to a portion of intact derma of the individual; and
transdermally delivering, for 24 hours or more, a systemic, therapeutically effective dose of the corticosteroid to the individual.

2. The method of claim 1, wherein the permeation enhancer is selected from the group consisting of fatty acids, esters of fatty acids, straight or branched chain alkyl alcohols and their esters, ethers and combinations thereof.

3. A transdermal patch for application to a portion of intact derma of an individual, the patch comprising:
a. a backing layer; and
b. a matrix adhesive layer, the matrix adhesive layer comprising in admixture:
i. a skin-compatible pressure-sensitive adhesive comprising an acrylate adhesive;
ii. a corticosteroid in an amount between 0.25% and 15% by weight based on the total weight of the matrix adhesive layer;
iii. one or more permeation enhancers that are fatty acids, fatty acid esters, C8-C16 alcohols (such as straight-chain alcohols), polyhydric alcohols whose hydroxyls are or are not alkylated with C1-C4 alkyls, cyclic or bicyclic compounds of C/H/O having 5 to 10 ring atoms of carbon or oxygen and two or more hydroxyls whose hydroxyls are or are not alkylated with C1-C4 alkyls, or branched compounds of C/H/O having 5 to 10 backbone atoms of carbon or oxygen and two or more hydroxyls whose hydroxyls are or are not alkylated with C1-C4 alkyls, wherein the permeation enhancer(s) are present in the matrix adhesive layer in an amount between 0.5% and 15% based on the total weight of the matrix adhesive layer;
the matrix adhesive layer, when applied to the intact derma of the individual, transdermally delivering, for 24 hours or more, a systemic, therapeutically effective dose of the corticosteroid to the individual.

4. The patch of claim 3, wherein the corticosteroid comprises dexamethasone.

5. The patch of claim 4, wherein the dexamethasone is present in an amount between 1% and 10% based on the total weight of the matrix adhesive layer.

6. The patch of claim 5, wherein the dexamethasone is present in an amount between 2.0% and 5.5% based on the total weight of the matrix adhesive layer.

7. The patch of claim 3, wherein the permeation enhancer is selected from oleic acid, isopropyl myristate, lauryl alcohol, dimethyl isosorbide, methyl oleate, and combinations thereof.

8. The patch of claim 7, wherein the permeation enhancer comprises oleic acid in an amount of between 0.5% and 15% based on the total weight of the matrix adhesive layer.

9. The patch of claim 8, wherein the permeation enhancer further comprises lauryl alcohol in an amount of between 0.5% and 15% based on the total weight of the matrix adhesive layer.

10. The patch of claim 8, wherein the permeation enhancer further comprises dimethyl isosorbide in an amount of between 0.5% and 15% based on the total weight of the matrix adhesive layer.

11. The patch of claim 3, wherein the patch delivers a daily dose of between 0.25 to 8.0 mg to the individual.

12. A method for the transdermal administration of a corticosteroid to an individual, the method comprising applying to the derma of the individual a patch according to claim 1, the method transdermally delivering, for 24 hours or more, a systemic, therapeutically effective dose of the corticosteroid to the individual.

13. The method of claim 12, wherein the corticosteroid comprises dexamethasone.

14. The method of claim 13, wherein the dexamethasone is present in an amount between 1% and 10% based on the total weight of the matrix adhesive layer.

15. The method of claim 14, wherein the dexamethasone is present in an amount between 2.0% and 5.5% based on the total weight of the matrix adhesive layer.

16. The method of claim 12, wherein the permeation enhancer is selected from oleic acid, isopropyl myristate, lauryl alcohol, dimethyl isosorbide, methyl oleate, and combinations thereof.

17. The method of claim 16, wherein the permeation enhancer comprises oleic acid in an amount of between 0.5% and 15% based on the total weight of the matrix adhesive layer.

18. The method of claim 17, wherein the permeation enhancer further comprises lauryl alcohol in an amount of between 0.5% and 15% based on the total weight of the matrix adhesive layer.

19. The method of claim 17, wherein the permeation enhancer further comprises dimethyl isosorbide in an amount of between 0.5% and 15% based on the total weight of the matrix adhesive layer.

20. The method of claim 12, wherein the patch delivers a daily dose of between 0.25 to 8.0 mg to the individual.
